(19) United States
(12) Patent Application Publication
(10) Pub. No.: US 2008/0162056 A1
(43) Pub. Date: Jul. 3, 2008

(54) METHOD AND APPARATUS FOR DETERMINATION OF GAS IN PLACE

(76) Inventor: Keith Howarth Greaves, Salt Lake City, UT (US)

Correspondence Address: DUKE W. YEE;YEE & ASSOCIATES, P.C., P.O. BOX 802333, DALLAS, TX, 75380, US

(21) Appl. No.: 11/618,392

(22) Filed: Dec. 29, 2006

(51) Int. Cl. G01N 30/00; G01N 33/00

(52) U.S. Cl. 702/24

(57) ABSTRACT

A computer implemented method, apparatus, and computer program product for predicting initial gas production in a well. A cumulative amount of each gas species present in a gas sample taken from the well is identified. A set of data points corresponding to the cumulative amount of each gas species present in a gas sample for each gas species is generated. A Y-intercept value for each gas species is calculated based on the set of data points. A projected initial amount of the given gas species produced from the well is presented. The projected initial amount of the given gas species produced from the well is determined using the Y-intercept value. The Y-intercept value indicates a cumulative amount of gas for a given gas species.

### BACKGROUND OF THE INVENTION

1. Field of the Invention

The present invention relates generally to identifying gas present in a formation and in particular, to a method and apparatus for analyzing gas sorption data. Still more particularly, the present invention relates to a computer implemented method, apparatus, and computer usable program code for analyzing data from a core sample to identify gas in place for a gas well.

2. Background of the Invention

In the production life cycle of natural resources, such as petroleum oil and natural gas, these types of resources are extracted from reservoir fields in geological formations. Different stages in this life cycle include exploration, appraisal, reservoir development, production decline, and abandonment of the reservoir. In these different phases, decisions are made to properly allocate resources to assure that the reservoir meets its production potential. In the early stages of this cycle, the distribution of internal properties within the reservoir is almost unknown. As development of the reservoir continues, different types of data regarding the reservoir are collected. This data includes, for example, gas sorption data from core samples, well logs, and production data. This information is combined to construct an understanding of the distribution of reservoir properties in the formation.

Natural gas is a fossil fuel consisting primarily of methane, also represented by the chemical formula “CH₄”. Natural gas may also include ethane “C₂H₆”, propane “C₃H₈”, butane “C₄H₁₀” and other organic gases. Natural gas may be found in oil fields, as well as in natural gas fields and coal beds. Natural gas found adsorbed into coal beds is sometimes referred to as coalbed methane. As used herein, adsorption refers to the process by which a gas accumulates on the surface of a solid or a liquid. The term desorption refers to the opposite process, in which gases are released from the surface of a solid or liquid.

The species of gases adsorbed on solids, such as coalbed methane, may be identified by taking a core sample and measuring the gases released from the core sample. The data obtained from the core sample may be analyzed to identify each species of gas present in a given gas well.

However, the approaches to analyzing data from well sites that are available today have some important disadvantages. Specifically, currently available techniques allow for analyzing and interpreting different types of data. For example, a program may allow for analysis and interpretation of porosity measurements to identify the species of gases present in a core sample and the amounts of gas released by the core sample. Analysis may also allow a user to identify the various gas species present in a gas well. However, currently available analysis methods and techniques do not permit a user to accurately or reliably predict the gas species composition or initial quantities of gas in place for each gas species in a given gas well.

### SUMMARY OF THE INVENTION

In view of the above problems, an object of the present invention is to provide a method, apparatus and computer program product for projecting initial gas production in a well while eliminating or minimizing the impact of the problems and limitations described. In one embodiment, a computer implemented method is provided for predicting initial gas production in a well. An amount of lost gas is extrapolated based on an amount of measured gas. The amount of lost gas is an amount of gas that was not measured. A cumulative amount of each gas species present in a gas sample taken from the well is identified. The cumulative amount of each gas species comprises a measured amount of gas and an extrapolated lost amount of gas.

A set of data points corresponding to the cumulative amount of each gas species present in a gas sample for each gas species is generated. A Y-intercept value for each gas species is calculated using the set of data points. The Y-intercept indicates a cumulative amount of gas for the given gas species. A projected initial amount of the given gas species produced from the well is presented based on the Y-intercept value, wherein the Y-intercept value indicates a cumulative amount of gas for a given gas species. A set of tests for processing gas in the well are identified based on the projected initial amount of the given gas species produced for each gas species present in the well. A set of isotherms for extracting one or more gas species from the well may also be identified based on the projected initial amount of the given gas species produced from the well.

In another embodiment, the method plots the set of data points corresponding to the cumulative amount of each gas species present in the gas sample to form a graph. A regression analysis may be used to plot the data points on the graph. The graph includes a set of curves. Each curve represents a different gas species released by the core sample. The area under each curve is integrated to determine a gas in place for each gas species. The gas in place is the amount of gas present in the core sample. A Y-intercept on the graph is identified for each gas species in the gas sample. The graph is outputted with the identified Y-intercepts indicating the projected amount of each given gas species produced from the well.

The illustrative embodiments also provide a computer program product. The computer program product includes a computer usable medium including computer usable program code for predicting initial gas production in a well. The computer program product provides computer usable program code for identifying a cumulative amount of each gas species present in a gas sample taken from the well; generating a set of data points corresponding to the cumulative amount of each gas species present in a gas sample for each gas species; calculating a Y-intercept value for each gas species using the set of data points; and presenting a projected initial amount of the given gas species produced from the well based on the Y-intercept value, wherein the Y-intercept value indicates a cumulative amount of gas for a given gas species. The computer program product also provides computer usable program code for identifying a set of isotherms to run based on the projected initial amount of the given gas species produced from the well; and identifying a set of tests for processing gas in the well based on the projected initial amount of the given gas species produced for each gas species present in the well.

In one embodiment, the computer program product also provides computer usable program code for plotting the set of data points corresponding to the cumulative amount of each gas species present in the gas sample to form a graph; identifying a Y-intercept on the graph for each gas species in the gas sample; and outputting the graph with the identified Y-intercepts indicating the projected amount of each given gas species produced from the well.

In another embodiment, the graph generated by the computer program product includes a set of curves. Each curve represents a different gas species released by the core sample. The computer usable program code integrates the area under each curve to determine a gas in place for each gas species, wherein the gas in place is the amount of gas present in the core sample.

The illustrative embodiments also provide a system for predicting initial gas production in a well. The system includes a container system for measuring an amount of gas released from a core sample to form a gas sample. The system also includes a gas chromatograph. The gas chromatograph identifies each species in the gas sample. The system also includes an analysis engine. The analysis engine identifies a cumulative amount of each gas species present in a gas sample taken from the well; plots a set of data points corresponding to the cumulative amount of each gas species present in a gas sample to form a graph; identifies a Y-intercept on the graph for a given gas species in the gas sample; and outputs a projected initial amount of the given gas species produced from the well based on the cumulated amount of gas indicated by the Y-intercept.

The illustrative embodiments also provide an apparatus for predicting initial gas production in a well. The apparatus includes means for identifying a cumulative amount of each gas species present in a gas sample taken from the well; means for generating a set of data points corresponding to the cumulative amount of each gas species present in a gas sample for each gas species; means for calculating a Y-intercept value for each gas species using the set of data points; and means for presenting a projected initial amount of the given gas species produced from the well based on the Y-intercept value. The Y-intercept value indicates an initial amount of gas for a given gas species produced from the well from which the gas sample was taken. This information regarding the initial gas produced by a given well is useful for determining what processes should be employed to extract a given gas species. This information may also be used to determine whether a well will be economical to produce.

Finally, the illustrative embodiments provide a method of improving well production by predicting initial gas production in a well. A cumulative amount of each gas species present in a gas sample taken from the well is identified. A set of data points corresponding to the cumulative amount of each gas species present in a gas sample for each gas species is generated. A Y-intercept value for each gas species is calculated using the set of data points. A projected initial amount of the given gas species produced from the well is presented based on the Y-intercept value. Next, the predicted initial gas production is used to implement production operations for a field containing the well.

In one embodiment, implementing production operations for the field containing the well includes determining whether gas extracted from the well will require treatment. In another embodiment, implementing production operations for the field containing the well includes drilling a number of offset wells. Implementing production operations for the field containing the well may also include providing for CO2 sequestration.

Other objects, features and advantages of the present invention will become apparent to those of skill in the art by reference to the figures, the description that follows and the claims.

### BRIEF DESCRIPTION OF THE DRAWINGS

FIG. 1 is a pictorial representation of a network data processing system in which a preferred embodiment of the present invention may be implemented;

FIG. 2 is a diagram illustrating a well site from which data is obtained in accordance with a preferred embodiment of the present invention;

FIG. 3 is a diagram illustrating a current canister system for measuring gas release from a core sample;

FIG. 4 is a diagram of a design system as depicted in accordance with a preferred embodiment of the present invention.

FIG. 5 depicts a block diagram illustrating a dataflow when core sample data is analyzed in accordance with a preferred embodiment of the invention;

FIG. 6 is a graph generated using a current method for depicting total measured gas release from a core sample over time;

FIG. 7 is a graph generated using a current method for depicting measured gas and extrapolated lost gas desorption using current methods;

FIG. 8 is a graph generated using current methods for characterizing gas species released from a core sample;

FIG. 9 is an illustrative example of a set of equations for calculating gas in place in accordance with a preferred embodiment of the present invention;

FIG. 10 is a graph illustrating gas in place in accordance with a preferred embodiment of the present invention; and

FIG. 11 is a flowchart of a process for identifying gas in place for gas stored in porosity in accordance with a preferred embodiment of the present invention.

### DETAILED DESCRIPTION OF THE DRAWINGS

In the following detailed description of the preferred embodiments and other embodiments of the invention, reference is made to the accompanying drawings. It is to be understood that those of skill in the art will readily see other embodiments and changes may be made without departing from the scope of the invention.

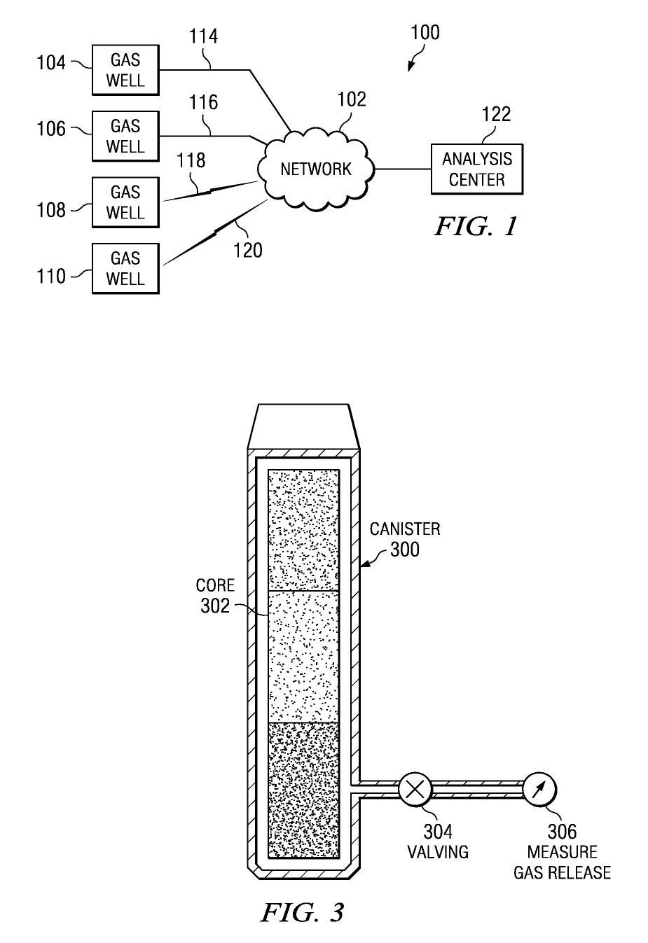

With reference now to FIG. 1, a pictorial representation of a network data processing system is depicted in which a preferred embodiment of the present invention may be implemented. In this example, network data processing system 100 is a network of computing devices in which different embodiments of the present invention may be implemented. Network data processing system 100 includes network 102, which is a medium used to provide communications links between various devices and computers in communication with each other within network data processing system 100. Network 102 may include connections, such as wire, wireless communications links, or fiber optic cables.

In this depicted example, gas wells 104, 106, 108, and 110 have computers or other computing devices that produce data regarding wells located at these well sites. In these examples, gas wells 104, 106, 108, and 110 are located in a geographic region. This geographic region is a single reservoir in these examples. Of course, these well sites may be distributed across diverse geographic regions and/or over multiple reservoirs, depending on the particular implementation.

Gas wells 104-110 may be wells that produce petroleum oil and natural gas or wells that produce only natural gas. In this example, gas wells 104-110 are gas wells in which natural gas is adsorbed into coals or carbonaceous shales. However, gas wells 104-110 may include gas wells in which gases are adsorbed into any coals, shales, porous organic solids, or other adsorption material. Gas wells in which natural gas is adsorbed into coals are sometimes referred to as coalbed methane wells.

Gas wells 104 and 106 have wired communications links 114 and 116 to network 102. Gas wells 108 and 110 have wireless communications links 118 and 120 to network 102. Communications links 114-120 may be used to transmit data regarding gas wells 104-110 to analysis center 122. Data regarding gas wells 104-110 may include data such as gas desorption rates measured from a core sample, data regarding adsorbed and desorbed gas phase on the organics of coals and shales associated with gas wells 104-110, and/or any other data regarding a given gas well.

Analysis center 122 is a location at which data processing systems, such as servers, clients, and/or any other computing devices for processing data collected from gas wells 104, 106, 108, and 110 are located. In this example, a single analysis center is depicted. However, depending on the particular implementation, multiple analysis centers may be present. These analysis centers may be located at an office or local to the geographic location at which gas wells 104-110 are located. In other words, analysis center 122 may be located locally to or remotely from a location of a gas well, such as gas wells 104-110.

In the depicted example, network data processing system 100 is the Internet with network 102 representing a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other computer systems that route data and messages. Of course, network data processing system 100 also may be implemented as a number of different types of networks, such as for example, an intranet, a local area network (LAN), or a wide area network (WAN). FIG. 1 is intended as an example, and not as an architectural limitation for different embodiments.

The different embodiments of the present invention provide a computer implemented method, apparatus, and computer usable program code for identifying gas species, determining adsorbed and desorbed gas phases on the organics of coals and shales, and identifying percentages of gas in place for each identified gas species. In these illustrative embodiments, the gas in place is determined based on desorption of gas on coals and shales using a modified Boyle's law green volume porosimeter and related data analysis. Desorption data is obtained using a standard canister system for measuring gas release from a core sample. Desorption data is analyzed to form a graph predicting initial gas in place for each identified gas species. This information may be used for better well production planning. In other words, the graph predicting initial gas species production may be used to more accurately select gas extraction and purification processes for a particular well.

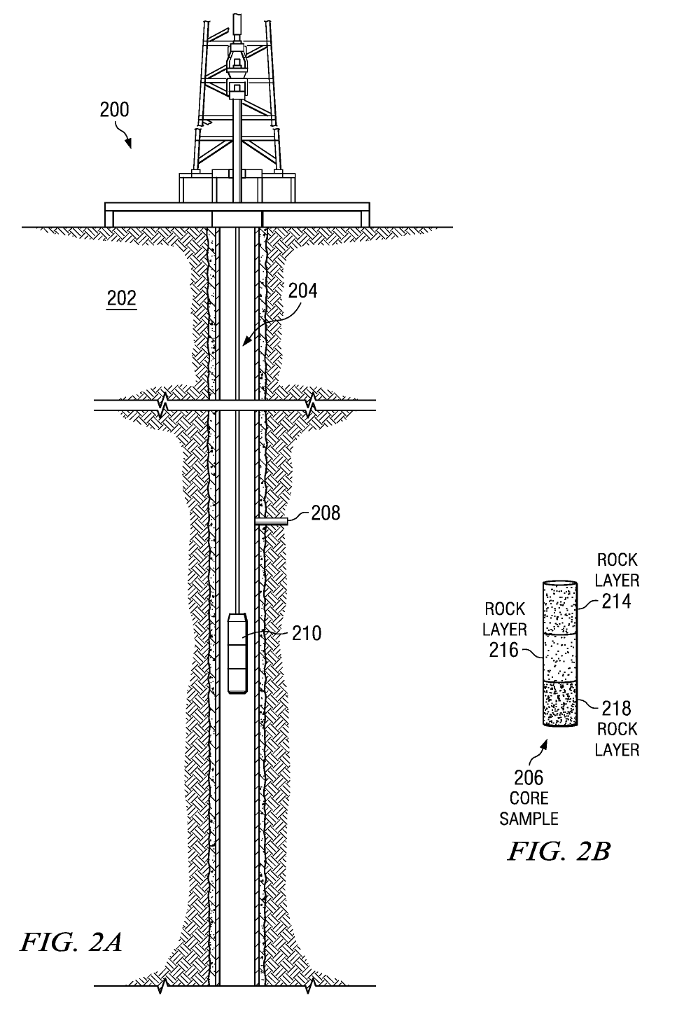

Turning now to FIG. 2, a diagram illustrating a well site from which data is obtained is depicted in accordance with a preferred embodiment of the present invention. Well site 200 is an example of a gas well site, such as gas well 104 in FIG. 1. In this example, well site 200 is located on formation 202. During the creations of wellbore 204 in formation 202, different samples are obtained. For example, core sample 206 may be obtained as well as sidewall plug 208. Further, logging tool 210 may be used to obtain other information, such as pressure measurements and factor information. Further, from creating wellbore 204, drill cuttings and mud logs are obtained. This information may be collected by a data processing system and transmitted to an analysis center, such as analysis center 122 in FIG. 1 for analysis.

In this example, wellbore 204 is a vertical wellbore. However, wellbore 204 may also include a wellbore that deviates from true vertical. In other words, wellbore 204 may not be a truly vertical well wellbore, but may also include a wellbore that is angled. Likewise, one or more portions of wellbore 204 may also be horizontal at one or more points or may start as a single vertical wellbore but split into multiple well paths at some depth below the surface, or may have any configuration known to those skilled in the art.

Core sample 206 is a sample of the coals, shales, and/or other organic solids taken from well site 200. Core sample 206 may include multiple layers of different types of organic solids, such as rock layers 214-218. Gas, such as natural gas, may be adsorbed into one or more of rock layers 214-218. Each rock layer may be a different type of coal, shale, or other rock. Thus, varying amounts of different gas species may be adsorbed into each rock layer in rock layers 216-218. However, core sample 206 may also consist of a single type of coal, shale, or other rock. In this example, rock layers 216-218 are coal layers.

For example, core sample 206 may be placed in a standard canister system used to measure gas release from core sample 206. The canister system measures gas desorption from core sample 206. In other words, core sample 206 releases gases. The released gases are captured and measured by the canister system. The gas desorption data measured by the canister system may be transmitted by data processing system 214 to an analysis center for further analysis. Also, images of core samples and other data taken by devices at well site 200 may be collected and sent to data processing system 214 for transmission to analysis center 122.

In this illustrative example, well site 200 provides continuous data and discrete data. The continuous data may be well site data or laboratory data and the discrete data also may be well site data or laboratory data in these examples. Well site data is data obtained through measurements made on the well while laboratory data is made from measurements obtained from samples from well site 200. For example, continuous well site data includes, for example, seismic, log/log suit and measurements while drilling. Continuous laboratory data includes, for example, strength profiles in core gamma information. Discrete well site data includes, for example, sidewall plugs, drill cuttings, pressure measurements, and gas flow detection measurements. The discrete laboratory data may include, for example, laboratory measurements made on plugs or cores obtained from well site 200. Of course, the different illustrative embodiments may be applied to any continuous well site data, continuous laboratory data, discrete well site data, and discrete laboratory data in addition to or in place of those illustrated in these examples.

This information may be input or entered into data processing system 214 for transmission to an analysis center for processing. Alternatively, depending on the particular implementation some or all processing of the data from well site 200 may be performed using data processing system 214. For example, data processing 214 may be used to preprocess the data or perform all of the analysis on the data from well site 200. If all the analysis is performed using data processing system 214 the results may then be transmitted to the analysis center to be combined from results from other well sites to provide a final result.

FIG. 3 is a diagram illustrating a current canister system for measuring gas release from a core sample. Canister 300 is any type of known or available standard canister system for measuring gas desorption from a core sample, such as core 302. Core 302 is a core sample taken from a gas well, such as core sample 206 in FIG. 2. Valving 304 is a valve to measure gas release 306 by canister 300. In other words, core 302 is placed inside canister 300. Core 302 evolves gases, including, but not limited to, gases such as carbon dioxide “CO₂”, methane, ethane, and/or nitrogen “N₂” gases. Only some of the gases adsorbed in a core sample will be desirable gases. For example, methane and ethane are desirable gases. However, carbon dioxide may not be desirable. Therefore, the amounts of each gas species evolved are measured in order to attempt to estimate the concentration of each gas in the particular gas well or coalbed.

Canister 300 measures the amounts of gases released by core 302 over time. Readings for the amount of gas released are taken over discrete time periods in the illustrative embodiments. For example, a user may take discrete readings regarding gas released from a core sample every ten minutes.

A user may manually determine the amount of gas release measured by canister 300 by manually reading valving 304. In another example, canister 300 may be coupled to a data processing system, such as data processing system 214 in FIG. 2. Data processing system 214 analyzes data regarding gas released by core 302 and provides this information to a user via a graphical user interface, a display device, a voice recognition system, or other output device.

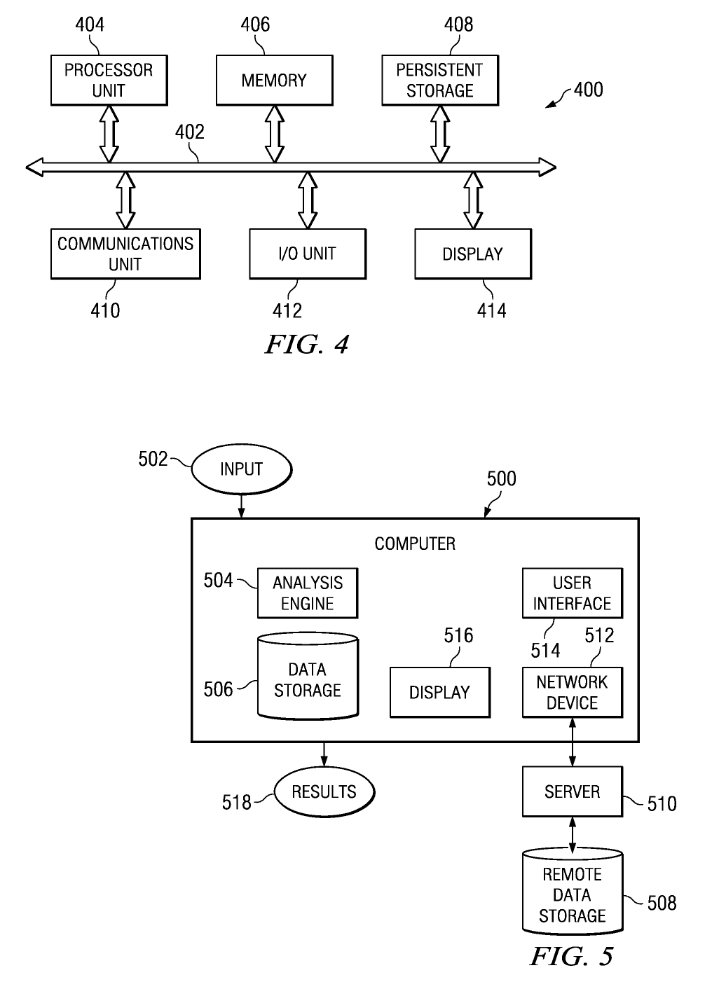

Turning now to FIG. 4, a diagram of a design system is depicted in accordance with a preferred embodiment of the present invention. In this illustrative example, data processing system 400 includes communications fabric 402, provides communications between processor unit 404, memory 406, persistent storage 408, communications unit 410, I/O unit 412, and display 414.

Processor unit 404 serves to execute instructions for software that may be loaded into memory 406. Processor unit 404 may be a set of one or more processors or may be a multi-processor core, depending on the particular implementation. Further processor unit 404 may be implemented using one or more heterogeneous processor systems in which a main processor is present with secondary processors on a single chip. Memory 406, in these examples, may be, for example, a random access memory. Persistent storage 408 may take various forms depending on the particular implementation. For example, persistent storage 408 may be, for example, a hard drive, a flash memory, a rewritable optical disk, a rewritable magnetic tape, or some combination of the above.

Communications unit 410, in these examples, provides for communications with other data processing systems or devices. In these examples, communications unit 410 is a network interface card. I/O unit 412 allows for input and output of data with other devices that may be connected to data processing system 400. For example, I/O unit 412 may provide a connection for user input though a keyboard and mouse. Further, I/O unit 412 may send output to a printer. Display 414 provides a mechanism to display information to a user.

Instructions for the operating system, the object-oriented programming system, and applications or programs are located on persistent storage 408. These instructions may be loaded into memory 406 for execution by processor unit 404. The processes of the different embodiments may be performed by processor unit 404 using computer implemented instructions, which may be located in a memory, such as memory 406.

Data processing system 400 may be implemented using any known or available computing device, including, but not limited to, a desktop computer, a laptop computer, a personal digital assistant (PDA), a tablet PC, or any other known or available computing device. In this example, data processing system 400 is a computing device in an analysis center, such as analysis center 122 in FIG. 2. In this example, data processing system 400 is a data processing system, such as data processing system 312 in FIG. 3. In accordance with the illustrative embodiments, data processing system 400 may be located locally to a wellbore or remotely to a wellbore.

Thus, the illustrative embodiments provide a computer implemented method, apparatus, and computer program product for predicting initial gas production in a well. A cumulative amount of each gas species present in a gas sample taken from the well is calculated. The cumulative amount of each gas species comprises a measured amount of gas and an extrapolated lost amount of gas. A set of data points corresponding to the cumulative amount of each gas species present in a gas sample is plotted to form a graph. As used herein, the term “set of” refers to a set of one or more items in a set. In this example, a set of data points includes one or more data points. The graph comprises a set of curves, and wherein each curve represents a different gas species released by the core sample.

A Y-intercept on the graph for a given gas species in the gas sample is identified. The Y-intercept indicates a cumulative amount of gas for the given gas species. A projected initial amount of the given gas species produced from the well based on the cumulative amount of gas indicated by the Y-intercept is output.

FIG. 5 depicts a block diagram illustrating a dataflow when core sample data is analyzed in accordance with a preferred embodiment of the invention. Computer 500 is a computing device for analyzing gas desorption data obtained by measuring gas evolution in a canister system, such as canister 300 in FIG. 3. Computer 500 also identifies a predicted initial gas in place for one or more gas species based on the gas desorption data. Computer 500 may be implemented using any type of computing device, such as a personal computer, laptop, personal digital assistant, or any other computing device. For example, computer 500 may be a computing device such as computer 400 in FIG. 4.

Input 502 is data regarding gas desorption from a core sample and/or any other data regarding gas sorption. Analysis engine 504 is a software component for analyzing input 502. Analysis engine 504 analyzes input 502 to identify gas species, amount of gas evolved from a given core sample, and/or identify a predicted initial gas production for a given gas well. A gas well may be a natural gas well, such as gas wells 104-110 in FIG. 1.

Data storage device 506 is any known or available device for storing data. Data storage device 506 may include a hard disk, a floppy disk, a flash memory, main memory, read-only memory (RAM), random access memory (ROM), nonvolatile random access memory (NV-RAM), or any other known or available device for storing data. In this example, data storage device 506 is located on or locally to, computer 500. However, data may also be stored on remote data storage 508. Remote data storage 508 is a data storage device located remotely to computer 500. Computer 500 accesses remote data storage 508 by means of server 510.

Server 510 is a server of any type of known or available server. Server 510 may be connected to a network. In the depicted example, server 510 provides data, such as boot files, operating system images, data files, and applications to computer 500.

Network device 512 is any type of network access software known or available for allowing computer 500 to access a network. Network device 512 connects to a network connection, such as network 102 in FIG. 1. The network connection permits access to any type of network, such as a local area network (LAN), a wide area network (WAN), or the Internet.

User interface 514 is any type of known or available interface for providing input to computer 500, including but not limited to, a graphical user interface (GUI), a menu-driven interface, a command line interface, a voice recognition system, a keyboard and mouse, a touch-screen, or any other type of interface to permit a user to enter data as input 502 and receive data as output. Display 516 is a display screen for displaying output to a user. In this example, display 516 is a separate component from user interface. However, in another embodiment, display 516 is a part of user interface 514.

Results 518 are the results of analyzing input 502 calculated by computer 500. Results may be provided to a user as output via user interface 514 and/or display 516. In this example, results 518 include a predicted initial gas in place of one or more gas species in a given gas well.

Thus, in this example, a user obtains a core sample from a gas well. The core sample is placed in a container for measuring gas evolution or gas release from the core sample. Data regarding gas release measured is entered into computer 500 as input 502. Analysis engine 504 identifies the total amount of gas release measured. Analysis engine 504 extrapolates an amount of lost gas that was not measured and/or an amount of residual gas remaining in the core sample. Residual gas is gas that is not released from a solid, such as coal, but may be obtained by crushing the solid.

Analysis engine 504 identifies each gas species and amounts of each gas species released by the core sample. Analysis engine 504 performs a series of equations in accordance with the illustrative embodiments of the present invention to identify a composition or gas in place for each gas species in the sample. Analysis engine 504 plots data points for cumulative gas of each species calculated by analysis engine 504 to form a graph illustrating gas in place. Analysis engine 504 identifies the Y-intercepts on the graph for each gas species. The cumulative gas value at a Y-intercept for a given gas species indicates a projected initial gas production for that given gas species from the given gas well. Analysis engine 504 stores results 518 of the gas in place analysis in data storage 506 and/or remote data storage 508. Analysis engine 504 may also output results 518 to a user via user interface 514 and/or display 516.

Results 518 indicate the composition of gas in the core sample. Results 518 also predict percentages of each gas species and initial amounts of gas produced by the gas well associated with the core sample for each gas species. A user may use results 518 to plan development and production of a given gas well. A user may determine which gas purification and retrieval methods to employ to retrieve desired gas species from the gas well.

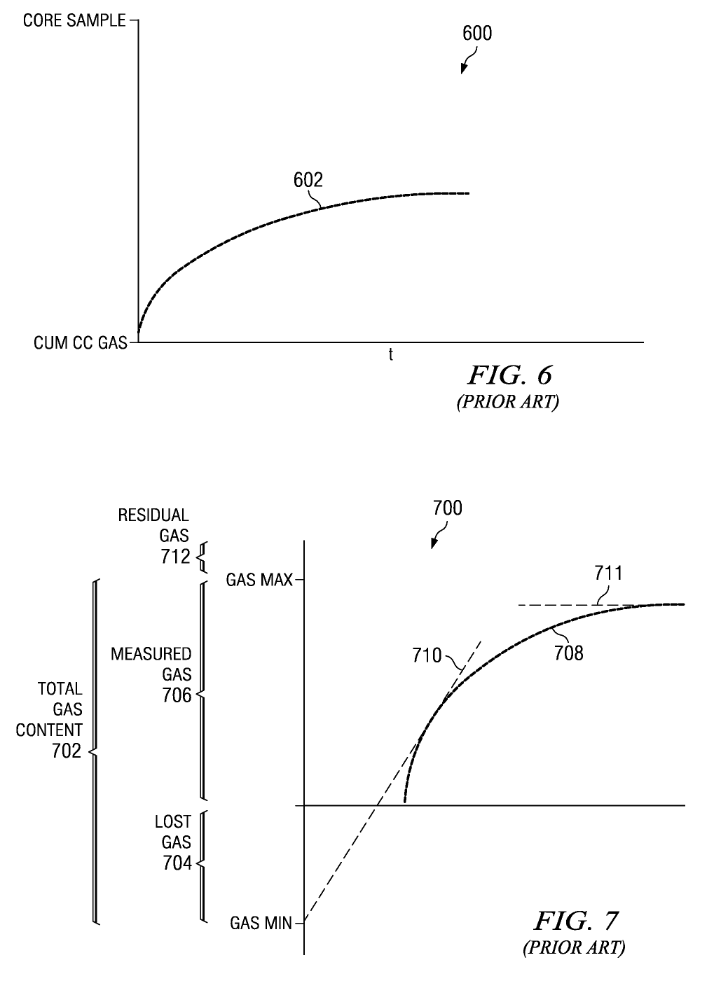

Referring now to FIG. 6, a graph generated using a current method for depicting total measured gas release from a core sample over time is shown. Graph 600 is a graph of total gas evolved from a given core sample over time. Graph 600 may be generated using current methods for plotting total gas evolved from a core sample over time. In this example, evolved gas is measured in cubic centimeters (cc) of gas. Curve 602 illustrates the measured amounts of total gas evolved from a core sample over time. Curve 602 begins at time zero (to).

However, curve 602 only depicts amounts of gas evolved from a core sample after the core sample was placed inside a canister system for measuring gas desorption, such as canister 300 in FIG. 3. Curve 602 does not identify amounts of discrete gas samples released from the core sample. Curve 602 also fails to provide data for reliably predicting an amount of gas in place in the reservoir from which the core sample was taken. In other words, curve 602 does not enable a user to accurately predict initial production of discrete gas species in a reservoir.

Moreover, curve 602 does not illustrate amounts of gas desorbed by the core sample prior to placement of the core sample into the canister for gas chromatography analysis. The unmeasured amount of gas evolved from the core sample prior to placement of the core sample in the canister is referred to as lost gas.

The equation of any straight line may be represented as y=mx+b. The slope of the line is represented by “m.” The Y-intercept is represented as “b.” A Cartesian coordinate plane is a plane having an x-axis and a Y-axis. A point can be identified by an x-value and a y-value, such as (x,y). When a straight line is represented on a two-dimensional Cartesian coordinate plane, the Y-intercept “b” is the value of y at a point where the straight line intersects the Y-axis of the coordinate plane. In other words, the Y-intercept is a point at which a function intersects the line at x=0.

FIG. 7 is a graph generated using a current method for depicting measured gas and extrapolated lost gas desorption using current methods. Graph 700 is a graph depicting total gas content 702. Total gas content 702 includes lost gas 704 and measured gas 706 released from a core sample. Lost gas 704 is the amount of gas evolved or released from the core sample before measurement of released gas began. In other words, lost gas 704 is the amount of gas released from the core sample without being measured. Measured gas 706 is the amount of gas released from the core sample that was actually measured.

Line 708 is a line showing the total measured gas over time. Lost gas 704 can be extrapolated by using any known or available process for extrapolating an amount of lost gas based on an amount of measured gas, including, but not limited to, a U.S. Bureau of mines technique used in this example to extrapolate a linear graph line 710 through a point on the graph at time zero (to) back to the y-intercept. The portion of line 710 from the Y-intercept to the point along the x-axis at time zero is the extrapolated amount of lost gas 704. Thus, the total amount of measured gas 706 and lost gas 704 is the total gas content desorbed from the core sample.

In graph 700, the Y-intercept correlating to the plateau of the curve is also determined to form line 711. Line 711 intercepts with the Y-axis at a point at which gas measured is a maximum value or “gas max.” The total gas content may be calculated as the minimum amount of gas or “gas min” subtracted from the maximum amount of measured gas. The “gas min” is the Y-intercept value for the amount of gas evolved. In this example, the total amount of gas released includes both measured gas and extrapolated lost gas. Thus, the total amount of gas released may be calculated follows:

Total gas content=gas max-gas min.

Residual gas 712 is the amount of gas that remains adsorbed on the core that fails to be desorbed. Residual gas 712 can be measured by crushing the core sample to force additional adsorbed gases to be released. The gas released after crushing the core sample is residual gas 712. Thus, curve 708 can be extended by crushing the rock in the core sample to obtain residual gas 712. The additional gas evolved from the crushed core sample can be added to the total gas content to obtain extended total gas content. The extended total gas content includes the lost gas, measured gas, and residual gas released from a given core sample over time. However, curve 708 only shows the total amounts of gas released from the core sample. Curve 708 does not distinguish amount of individual species of gas released. In other words, curve 708 can illustrate the total amount of gas released at a given time, but it does not illustrate the amount of each individual gas released by a core sample containing multiple different species of desorbing gases. Moreover, curve 708 cannot be used to determine a projected initial gas production for one or more gas species in a gas well.

In another embodiment, after gas releases during desorption, the core sample is not crushed to release residual gas. Instead, adsorption may be performed to determine how much gas the core sample can accept. Gases may be added to the canister containing the core sample to identify how much of the gas can be adsorbed by the core sample.

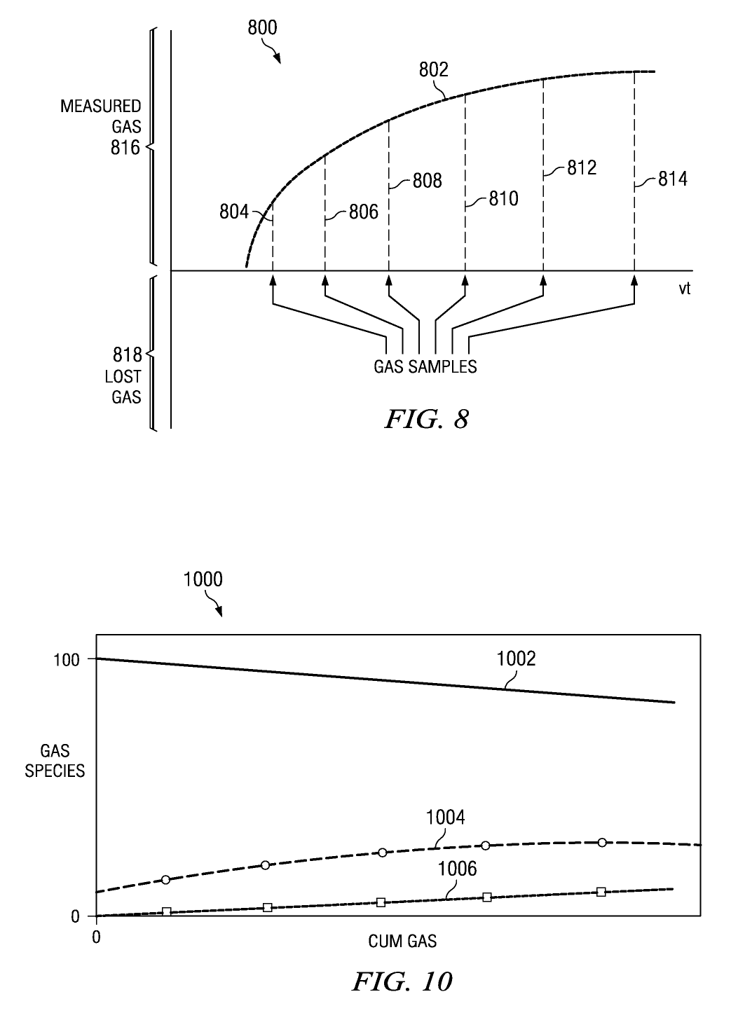

Referring now to FIG. 8, a graph generated using current methods for characterizing gas species released from a core sample. Gas chromatography refers to a chemical analysis for separating and identifying different gas species in a sample containing multiple gases. Any known or available gas chromatography may be used to separate and identify gas species desorbed from a core sample in a standard container system for measuring gas evolution.

Graph 800 is a graph showing amounts of gases released over time. Graph 800 may be generated using gas chromatography to separate discrete gas species from a sample containing multiple different gas species. Curve 802 illustrates an amount of gas evolved from a core sample over a square root of time. The measurements taken during the desorption process are analyzed using a gas chromatograph to identify gas species and determine gas composition in the core sample.

In gas chromatography, different gases pass through a given gas chromatograph at different rates depending on the type of gas and the chemical composition of the gas. Thus, the area under curve 802 can be used to identify disparate gas species based on gas evolution over the square root of time. In this example, area 804-814 may represent disparate gas species in the core sample. In other words, area 804 may represent one gas species while area 806 may represent a gas species with a different chemical composition and thus, a different rate of movement through the gas chromatograph. The data obtained from the gas chromatograph and illustrated in graph 800 may be used to identify each gas species present in the core sample.

Measured gas 816 is the amount of gas evolved from a core sample that was actually measured. Lost gas 818 is an amount of gas evolved from the core sample that was not measured. In this example, lost gas 818 is an amount of gas released from the core sample prior to the core sample being placed in a canister for measurement of gas evolution. Lost gas 818 can be extrapolated based on measured gas 816, as illustrated in FIG. 7 above. The canister used may be any type of canister for measuring gas evolution, including, but not limited to, a canister such as canister 300 in FIG. 3. In this example, the canister includes a gas chromatograph.

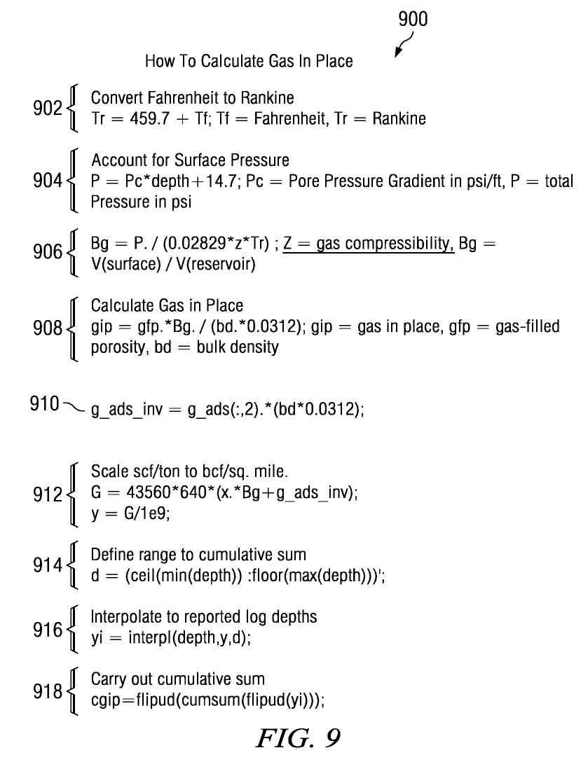

FIG. 9 is an illustrative example of a set of equations for calculating gas in place in accordance with a preferred embodiment of the present invention. Equations 900 are equations for converting gas sampling and analysis of gas species data obtained from a core sample, such as gas sampling and analysis data shown in FIG. 8, into data showing gas composition for gas stored in porosity of a given gas well from which the core sample was obtained.

Section 902 provides a formula for converting temperature in Fahrenheit to a temperature unit on the Rankine (Tr) temperature scale. In the Rankin scale, zero degrees (0°) Rankine is equal to −459.7 degrees in Fahrenheit (Tf). The formula to convert from Fahrenheit to Rankine is as follows:

Rankine (Tr)=459.7+Farhenheit (Tf).

Section 904 is a formula for taking into account surface pressure. The total pressure (P)in pounds per square inch (psi) is equal to the pore pressure gradient (Pc) multiplied by the depth and added to 14.7. The formula is as follows:

P=Pc*depth+14.7.

When hydrocarbons in a well are transferred from the reservoir to the surface, the pressure on the hydrocarbons will decrease. As a result of the decreasing pressure, the volume of the hydrocarbons will also change. The gas compressibility factor is represented by the variable “Bg”. Section 906 illustrates equations for taking into account gas compressibility. As shown in section 906, “Bg” is equal to the surface volume divided by the reservoir volume. The gas compressibility factor is represented by the variable “z”. The gas shrinkage factor “Bg” for changing volume of hydrocarbons can be calculated in accordance with the following equation:

Bg=P./(0.028929*z*Tr).

Section 908 provides an equation for calculating gas in place “gip”. The gas in place is equal to gas-filled porosity multiplied by the gas compressibility factor to find a value. This value is then divided by bulk density multiplied by 0.0312. In other words, gas in place may be calculated as follows:

gip=gfp*Bg./(bd*0.0312).

The cumulative sum is calculated and added to the adsorbed component in section 910 as follows:

g_{—}ads_{—}inv×g_{—}ads(:,2).*(bd*0.0312).

The variable “G” in section 912 is calculated from gfp*Bg. Therefore, the adsorbed scf/ton is transformed into the same variable and added to the free gas version. Adsorbed gas (g_ads) calculated separately from TOC, V1, and P1 based on isotherms. An exemplary formula for scaling scf/ton to bcf/square mile is as follows:

G=43560*640*(x.*Bg+g_{—}ads_{—}inv) where y=G/le9.

Section 912 provides an exemplary formula for defining a range to cumulative sum “d”. The formula states:

d=(ceil(min(depth)):floor(max(depth))′.

Section 914 provides an equation for defining a range to cumulative sum.

The equation is as follows:

D=(ceil(min(depth)):floor(maz(depth)))′;

An exemplary formula for interpolating to reported log depths at section 916 is as follows:

yi=interp1(depth,y,d).

The cumulative sum “cumsum” is carried out using the exemplary formula at section 918 which states as follows:

cgip=flipud(cumsum(flipud (yi))).

The calculations from the exemplary equations shown in 900 may be used as input to create a graph illustrating gas composition for gas stored in porosity in a given gas well. The gas in place (gip) indicates gas stored in porosity.

FIG. 10 is a graph illustrating gas in place in accordance with a preferred embodiment of the present invention. Graph 1000 shows a plot of the individual gas species based on gas composition. The data is plotted as cumulative gas along the X-axis versus percentage composition of each gas species. Cumulative gas may be calculated as shown in FIG. 9. In other words, graph 1000 shows a total gas released based on data shown in FIG. 9.

Equations are fit to the data points using regression analysis, such as those in Excel® programs available from Microsoft®. The area under each curve is integrated to determine or derive gas species composition in place. The Y-intercepts are used to estimate the initial gas production projected for the given well. In accordance with this embodiment, initial gas production can be accurately predicted using the method of this preferred embodiment within one to two percent of actual production.

The X-axis shows in situ gas content ranging from 0-100%. The cumulative gas for each gas species is shown along the Y-axis. Cumulative gas is the measured gas plus lost gas for a gas species. The Y-intercept is the point on graph 1000 at which cumulative gas for a given gas species is zero. In this example, line 1002 is a line representing cumulative gas for the gas species methane. Line 1004 is a line representing cumulative gas for the species carbon dioxide. Line 1006 represents cumulative gas for the gas species ethane. The gas stored in porosity within the gas well from which the core sample was taken can be determined for each gas species based on graph 1000. In other words, the initial gas production for methane in the gas well can be determined by calculating the Y-intercept for cumulative methane gas at line 1002.

Thus, future initial gas species production can be predicted in accordance with this advantageous embodiment by looking at the Y-intercept for the gas species through a backward extrapolation. The identification of initial gas production for each species present in a reservoir, such as a gas well, indicates if a user should treat the gas for extraction and production.

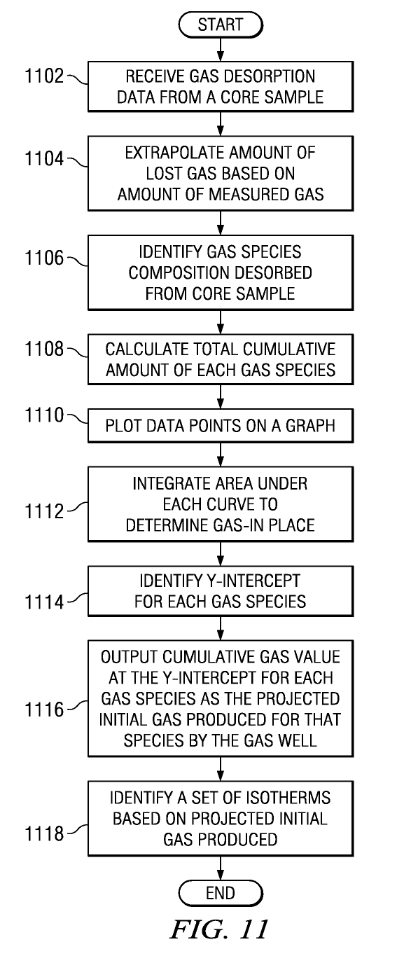

FIG. 11 is a flowchart of a process for identifying gas in place for gas stored in porosity in accordance with a preferred embodiment of the present invention. From the Y-intercepts, the gas composition for gas stored in porosity can be calculated. In this illustrative example in FIG. 11, the process may be implemented by a software component for analyzing gas release data obtained from a core sample, such as analysis engine 504 in FIG. 5. However, one or more of the steps illustrated in FIG. 11 may also be performed in whole or in part by a human user.

The process begins by receiving gas desorption data from a core sample (step 1102). Gas desorption data is data regarding gas released or evolved from a core sample. Gas desorption data may be obtained by a container system for measuring gas release, a gas chromatograph, and/or any other data for measuring gas desorption and identifying gas species. The process extrapolates an amount of lost gas based on the amount of measured gas (step 1104). The process then characterizes gas species composition desorbed from the core sample (step 1106). In other words, the process identifies each species of gas released from the core sample. The process then calculates a total cumulative amount of each gas released from the core sample (step 1108). The total cumulative amount of each gas is the measured gas amount and the lost gas amount.

The process plots data points corresponding to the species and cumulative amount of each gas species on a graph (step 1110). In order to plot the points, a set of data points corresponding to the cumulative amount of each gas species present in a gas sample is generated. In one embodiment, the process plots the data points using regression analysis. The process then integrates the area under each curve representing each gas species to determine a gas in place for each species (step 1112). The process identifies the Y-intercepts for each gas species on the graph (step 1114). The process then outputs the cumulative gas value at the Y-intercept for each gas species as the projected initial gas production for the respective gas species (step 1116). In other words, the cumulative gas value at the Y-intercept of a curve for a given gas species indicates the projected initial amount of gas that will be produced by the gas well from which the core sample was taken. The projected initial amount of gas to be produced calculated in accordance with this example may be accurate to within one or two percent. Finally, the process identifies a set of isotherms to run based on the projected initial amount of the given gas species produced from the well (step 1118) with the process terminating thereafter. Isotherms are used for pure gas adsorption. One or more isotherms may be selected to extract gas from a reservoir based on the predicted initial gas production for the gas species present in the reservoir. Using individual pure gas adsorption isotherm parameters (Vₘ,bᵢ) and calculated partial pressures from Y-intercepts for each gas species, a user can calculate adsorbed gas phase for each species using an extended Langmuir Isotherm.

Thus, the illustrative embodiments provide a computer implemented method, apparatus, and computer program product for predicting initial gas production in a well. A cumulative amount of each gas species present in a gas sample taken from the well is calculated. The cumulative amount of each gas species comprises a measured amount of gas and an extrapolated lost amount of gas. A set of data points corresponding to the cumulative amount of each gas species present in a gas sample is plotted to form a graph. The graph comprises a set of curves, and wherein each curve represents a different gas species released by the core sample.

A Y-intercept on the graph for a given gas species in the gas sample is identified. The Y-intercept indicates a cumulative amount of gas for the given gas species. A projected initial amount of the given gas species produced from the well based on the cumulated amount of gas indicated by the Y-intercept is output.

A user may select isotherms and tests for isolating and retrieving gas species from the well based on the projected initial amount of each given gas species produced by the well. This is an important advantage because the type of isotherm selected may be dependent upon the types of gas species present in the reservoir, as well as the total gas in place for each gas species. In this manner, a user may more efficiently plan and develop a gas well to reduce costs and increase production.

In other words, the predicted initial gas production identified by the illustrative embodiments is used to implement production operations for a field containing the well. Implementing production operations for the field containing the well includes drilling a number of offset wells. Implementing production operations may also include determining whether disposal of non-useful gas will be necessary. For example, predicted initial gas production data may be used to determine if it will be necessary to provide for CO2 sequestration during gas production. The initial gas production data may also indicate whether the gas contains hydrogen sulfide. Hydrogen sulfide is a poisonous, corrosive gas which can require special completion techniques and safety precautions.

Initial gas production data may also be used for determining whether gas will need treatment. If the gas does need treatment for production, the initial gas production information can be used to determine what types of gas treatments are appropriate. Finally, initial gas production data may also be used to determine whether to complete the well. For example, initial gas production data may indicate whether there is a sufficient quantity of sweet gas present in a given well to warrant completion of the well. In other words, a determination may be made as to whether it would be economical to complete a well based on the projected initial production amounts for each gas species present in the well.

The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved.

Although the foregoing is provided for purposes of illustrating, explaining and describing certain embodiments of the invention in particular detail, modifications and adaptations to the described methods, systems and other embodiments will be apparent to those skilled in the art and may be made without departing from the scope or spirit of the invention.

What is claimed is:

1. A computer implemented method for predicting initial gas production in a well, the computer implemented method comprising:
identifying a cumulative amount of each gas species present in a gas sample taken from the well;
generating a set of data points corresponding to the cumulative amount of each gas species present in a gas sample for each gas species;
calculating a Y-intercept value for each gas species using the set of data points; and
presenting a projected initial amount of the given gas species produced from the well based on the Y-intercept value.

2. The computer implemented method of claim 1 wherein the Y-intercept indicates a cumulative amount of gas for the given gas species.

3. The computer implemented method of claim 1 further comprising:
plotting the set of data points corresponding to the cumulative amount of each gas species present in the gas sample to form a graph;
identifying a Y-intercept on the graph for each gas species in the gas sample; and
outputting the graph with the identified Y-intercepts indicating the projected amount of each given gas species produced from the well.

4. The computer implemented method of claim 2 further comprising:
extrapolating an amount of lost gas based on the measured gas, wherein the amount of lost gas is an amount of gas that was not measured.

5. The computer implemented method of claim 1 further comprising:
identifying a set of tests for processing gas in the well based on the projected initial amount of the given gas species produced for each gas species present in the well.

6. The computer implemented method of claim 1 wherein the cumulative amount of each gas species comprises a measured amount of gas and an extrapolated lost amount of gas.

7. The computer implemented method of claim 1 wherein plotting the data points further comprises:
using regression analysis to plot the data points.

8. The computer implemented method of claim 1 wherein the graph comprises a set of curves, and wherein each curve represents a different gas species released by the core sample.

9. The computer implemented method of claim 7 wherein plotting the data points further comprises:
integrating the area under each curve to determine a gas in place for each gas species, wherein the gas in place is the amount of gas present in the core sample.

10. The computer implemented method of claim 1 further comprising:
identifying a set of isotherms for extracting one or more gas species from the well based on the projected initial amount of the given gas species produced from the well.

11. A computer program product comprising:
a computer usable medium including computer usable program code for predicting initial gas production in a well, said computer program product comprising:
computer usable program code for identifying a cumulative amount of each gas species present in a gas sample taken from the well;
computer usable program code for generating a set of data points corresponding to the cumulative amount of each gas species present in a gas sample for each gas species;
computer usable program code for calculating a Y-intercept value for each gas species using the set of data points; and
computer usable program code for presenting a projected initial amount of the given gas species produced from the well based on the Y-intercept value.

12. The computer program product of claim 11 wherein the Y-intercept indicates a cumulative amount of gas for the given gas species.

13. The computer program product of claim 11 further comprising:
computer usable program code for identifying a set of isotherms for extracting one or more gas species from the well based on the projected initial amount of the given gas species produced from the well.

14. The computer program product of claim 11 further comprising:
computer usable program code for identifying a set of tests for processing gas in the well based on the projected initial amount of the given gas species produced for each gas species present in the well.

15. The computer program product of claim 11 further comprising:
computer usable program code for plotting the set of data points corresponding to the cumulative amount of each gas species present in the gas sample to form a graph;
computer usable program code for identifying a Y-intercept on the graph for each gas species in the gas sample; and
computer usable program code for outputting the graph with the identified Y-intercepts indicating the projected amount of each given gas species produced from the well.

16. The computer program product of claim 11 wherein the graph comprises a set of curves, and wherein each curve represents a different gas species released by the core sample.

17. The computer program product of claim 16 further comprising:
computer usable program code for integrating the area under each curve to determine a gas in place for each gas species, wherein the gas in place is the amount of gas present in the core sample.

18. A system for predicting initial gas production in a well, the system comprising:
a container system, wherein the container system measures an amount of gas released from a core sample to form a gas sample;
a gas chromatograph, wherein the gas chromatograph identifies each species in the gas sample; and
an analysis engine, wherein the analysis engine identifies a cumulative amount of each gas species present in a gas sample taken from the well; plots a set of data points corresponding to the cumulative amount of each gas species present in a gas sample to form a graph; identifies a Y-intercept on the graph for a given gas species in the gas sample; and outputs a projected initial amount of the given gas species produced from the well using the cumulated amount of gas indicated by the Y-intercept.

19. The system of claim 19, wherein the Y-intercept indicates a cumulative amount of gas for the given gas species.

20. An apparatus for predicting initial gas production in a well, the apparatus comprising:
means for identifying a cumulative amount of each gas species present in a gas sample taken from the well;
means for generating a set of data points corresponding to the cumulative amount of each gas species present in a gas sample for each gas species;
means for calculating a Y-intercept value for each gas species using the set of data points; and
means for presenting a projected initial amount of the given gas species produced from the well based on the Y-intercept value, wherein the Y-intercept value indicates a cumulative amount of gas for a given gas species.

21. A method of improving well production by predicting initial gas production in a well, the method comprising:
identifying a cumulative amount of each gas species present in a gas sample taken from the well;
generating a set of data points corresponding to the cumulative amount of each gas species present in a gas sample for each gas species;
calculating a Y-intercept value for each gas species using the set of data points;
presenting a projected initial amount of the given gas species produced from the well based on the Y-intercept value; and
using the predicted initial gas production to implement production operations for a field containing the well.

22. The method of claim 21 wherein implementing production operations for the field containing the well includes determining whether gas extracted from the well will require treatment

23. The method of claim 21 wherein implementing production operations for the field containing the well includes drilling a number of offset wells.

24. The method of claim 21 wherein implementing production operations for the field containing the well includes providing for CO2 sequestration.
